(19) United States
(12) Patent Application Publication
(10) Pub. No.: US 2018/0267067 A1
(43) Pub. Date: Sep. 20, 2018

(54) METHODS FOR BIOANALYSIS OF 6-DIAZO-5-OXO-L-NORLEUCINE (DON) AND OTHER GLUTAMINE ANTAGONISTS

(71) Applicant: THE JOHNS HOPKINS UNIVERSITY, Baltimore, MD (US)

(72) Inventors: BARBARA S. SLUSHER, KINGSVILLE, MD (US); JESSE ALT, NOTTINGHAM, MD (US); CAMILO ROJAS, BALTIMORE, MD (US)

(73) Assignee: THE JOHNS HOPKINS UNIVERSITY, BALTIMORE, MD (US)

(21) Appl. No.: 15/542,164

(22) PCT Filed: Jan. 11, 2016
(86) PCT No.: PCT/US16/12868

§ 371 Date: Jul. 7, 2017

Related U.S. Application Data
(60) Provisional application No. 62/101,685, filed on Jan. 9, 2015.

(51) Int. Cl. G01N 33/94

(52) U.S. Cl. CPC G01N 33/94; C07D 207/22

(57) ABSTRACT

The presently disclosed subject matter provides methods for quantifying levels of glutamine antagonists, such as 6-diazo-5-oxo-L-norleucine (DON), including such glutamine antagonists resulting from in vivo conversion of ester prodrugs of such glutamine antagonists, in a biological sample.

### CROSS-REFERENCE TO RELATED APPLICATIONS

This application claims the benefit of U.S. Provisional Application No. 62/101,685, filed Jan. 9, 2015, which is incorporated herein by reference in its entirety.

### FEDERALLY SPONSORED RESEARCH OR DEVELOPMENT

This invention was made with government support under grant nos. R03DA032470 and P30MH075673-06 awarded by the National Institutes of Health (NIH). The government has certain rights in the invention.

### BACKGROUND

Glutamine is one of the most abundant amino acids in the human body and plays a critical role in cell growth, cell metabolism, and neurotransmission. Dysregulation of glutamine utilizing pathways has been associated with a variety of pathologies including cancer and neurodegenerative disease. Glutamine serves as a nitrogen donor for purine and pyrimidine production, which is required for de novo nucleotide synthesis (J. G. Cory and A. H. Cory, Critical roles of glutamine as nitrogen donors in purine and pyrimidine nucleotide synthesis: asparaginase treatment in childhood acute lymphoblastic leukemia, In Vivo. 20 (2006) 587-589).

Since de novo synthesis of nucleotides is upregulated to support DNA replication and RNA expression for rapid growth and division of cancer cells (X. Tong, F. Zhao, and C. B. Thompson, The molecular determinants of de novo nucleotide biosynthesis in cancer cells, Curr. Opin. Genet. Dev. 19 (2009) 32-37), inhibition of amidotransferases, the enzymes involved in the transfer of the amide group of glutamine to other molecules to initiate nucleotide synthesis, has been suggested as a potential cancer therapy.

Glutamine also is a major source of energy for neoplastic cells via glutaminolysis where glutaminase converts glutamine to glutamate which is further converted to α-ketoglutarate to enter the citric acid cycle (R. J. DeBerardinis and T. Cheng, Q's next: the diverse functions of glutamine in metabolism, cell biology and cancer, Oncogene. 29 (2010) 313-324). In support of this hypothesis, glutaminase inhibition has been shown to be efficacious in models of cancer (D. L. Kisner et al., The rediscovery of DON (6-diazo-5-oxo-L-norleucine), Recent Results Cancer Res. 74 (1980) 258-263; A. Le et al., Glucose-independent glutamine metabolism via TCA cycling for proliferation and survival in B cells, Cell Metab. 15 (2012) 110-121).

Further, glutaminase-catalyzed hydrolysis of glutamine to glutamate is a major source of glutamate in the brain (C. M. Thanki et al., In vivo release from cerebral cortex of [14C] glutamate synthesized from [U-14C] glutamine, J. Neurochem. 41 (1983) 611-617). Normal synaptic transmission in the central nervous system (CNS) involves the use of glutamate as the major excitatory amino acid neurotransmitter. Under certain pathological conditions, excessive glutamatergic signaling, termed excitotoxicity (P. Marmiroli and G. Cavaletti, The glutamatergic neurotransmission in the central nervous system, Curr. Med. Chem. 19 (2012) 1269-1276), is postulated to cause CNS damage in several neurodegenerative diseases, such as stroke (T. W. Lai et al., Excitotoxicity and stroke: identifying novel targets for neuroprotection, Prog. Neurobiol. 115 (2013) 157-188), amyotrophic lateral sclerosis (S. Vucic and M. C. Kiernan, Utility of transcranial magnetic stimulation in delineating amyotrophic lateral sclerosis pathophysiology, Handb. Clin. Neurol. 116 (2013) 561-575), Huntington's disease (M. D. Sepers and L. A. Raymond, Mechanisms of synaptic dysfunction and excitotoxicity in Huntington's disease, Drug Discov. Today (2014)), Alzheimer's disease (M. R. Hynd et al., Glutamate-mediated excitotoxicity and neurodegeneration in Alzheimer's disease, Neurochem. Int. 45 (2004) 583-595) and HIV associated dementia (M. C. Potter et al., Targeting the glutamatergic system for the treatment of HIV-associated neurocognitive disorders, J. Neuroimmune Pharmacol. 8 (2013) 594-607).

Consequently, inhibition of glutaminase has been suggested as a possible way to ameliorate high levels of glutamate in neurodegenerative diseases. In support of this hypothesis, glutaminase inhibition has been efficacious in models of CNS neurodegeneration (M. C. Potter et al., Targeting the glutamatergic system for the treatment of HIV-associated neurocognitive disorders, J. Neuroimmune Pharmacol. 8 (2013) 594-607; C. J. Chen et al., Glutamate released by Japanese encephalitis virus-infected microglia involves TNF-alpha signaling and contributes to neuronal death, Glia. 60 (2012) 487-501; A. R. Jayakumar et al., Glutamine in the mechanism of ammonia-induced astrocyte swelling, Neurochem. Int. 48 (2006) 623-628; I. Maezawa and L. W. Jin, Rett syndrome microglia damage dendrites and synapses by the elevated release of glutamate, J. Neurosci. 30 (2010) 5346-5356; A. G. Thomas et al., Small molecule glutaminase inhibitors block glutamate release from stimulated microglia, Biochem. Biophys. Res. Commun. 443 (2014) 32-36; C. Tian et al., HIV-infected macrophages mediate neuronal apoptosis through mitochondrial glutaminase, J. Neurochem. 105 (2008) 994-1005).

6-Diazo-5-oxo-L-norleucine (DON) is an amino acid analog of glutamine that is an inhibitor of glutamine utilizing enzymes, such as glutaminase, 2-N-amidotransferase, L-asparaginase and several enzymes involved in pyrimidine and purine de novo synthesis. DON inhibits 2-N-amidotransferase to block purine synthesis (D. L. Kisner et al., The rediscovery of DON (6-diazo-5-oxo-L-norleucine), Recent Results Cancer Res. 74 (1980) 258-263). DON was one of the earliest inhibitors to be identified for glutaminase (S. C. Hartman and T. F. McGrath, Glutaminase A of Escherichia coli. Reactions with the substrate analog, 6-diazo-5-oxonorleucine, J. Biol. Chem. 248 (1973) 8506-8510). It binds to the active site of glutaminase in an irreversible manner (S. C. Hartman and T. F. McGrath, Glutaminase A of Escherichia coli. Reactions with the substrate analog, 6-diazo-5-oxonorleucine, J. Biol. Chem. 248 (1973) 8506-8510; R. A. Shapiro et al., Inactivation of rat renal phosphate-dependent glutaminase with 6-diazo-5-oxo-L-norleucine. Evidence for interaction at the glutamine binding site, J. Biol. Chem. 254 (1979) 2835-2838; K. Thangavelu et al., Structural basis for the active site inhibition mechanism of human kidney-type glutaminase (KGA), Sci. Rep. 4 (2014) 3827).

As an inhibitor of glutamine metabolizing pathways, DON has been used both as a tool compound in preclinical in vivo models and also as a clinical candidate. There have been several clinical trials using DON (R. H. Earhart et al., Phase I trial of 6-diazo-5-oxo-L-norleucine (DON) administered by 5-day courses, Cancer Treat Rep. 66 (1982) 1215-1217; J. S. Kovach et al., Phase I and pharmacokinetic studies of DON, Cancer Treat Rep. 65 (1981) 1031-1036; G. Lynch et al., Phase II evaluation of DON (6-diazo-5-oxo-L-norleucine) in patients with advanced colorectal carcinoma, Am J Clin Oncol. 5 (1982) 541-543; J. Rubin, S. Sorensen, et al., A phase II study of 6-diazo-5-oxo-L-norleucine (DON, NSC-7365) in advanced large bowel carcinoma, Am. J. Clin. Oncol. 6 (1983) 325-326; R. B. Sklaroff et al., Phase I study of 6-diazo-5-oxo-L-norleucine (DON), Cancer Treat Rep. 64 (1980) 1247-1251; M. P. Sullivan et al., Pharmacokinetic and phase I study of intravenous DON (6-diazo-5-oxo-L-norleucine) in children, Cancer Chemother. Pharmacol. 21 (1988) 78-84); unfortunately it was not well tolerated at efficacious doses and has a narrow therapeutic window.

Recently, phase II clinical trials were reported for DON in combination with pegylated glutaminase with the goal of improving efficacy by co-administration with the glutamine depleting enzyme (C. Mueller et al., A phase IIa study of PEGylated glutaminase (PEG-PGA) plus 6-diazo-5-oxo-L-norleucine (DON) in patients with advanced refractory solid tumors, J. Clin. Oncol. 26 (2008) 2533). DON is still commonly used as a tool compound in glutamine-related research due to its solubility and efficacy in various in vivo models (B. B. Cao et al., The hypothalamus mediates the effect of cerebellar fastigial nuclear glutamatergic neurons on humoral immunity, Neuro. Endocrinol. Lett. 33 (2012) 393-400; L. M. Shelton et al., Glutamine targeting inhibits systemic metastasis in the VM-M3 murine tumor model, Int. J. Cancer. 127 (2010) 2478-2485). DON, with its polar structure and reactive moiety, however, would be expected to have difficulty reaching its target. Thus, a quantification assay for DON is of interest when using DON in animal models.

DON quantification has been carried out in the past by several methods that include HPLC of derivatized DON followed by absorbance and fluorescence detection (G. Powis and M. M. Ames, Determination of 6-diazo-5-oxo-L-norleucine in plasma and urine by reversed-phase high-performance liquid chromatography of the dansyl derivative, J. Chromatogr. 181 (1980) 95-99), ion-paired HPLC followed by absorbance detection (J. A. Nelson and B. Herbert, Rapid Analysis of 6Diazo5-oxo-L-norleucine (DON) in Human Plasma and Urine, J. Liquid Chromatogr. & Related Technologies 4(1981) 1641-1649), radioisotope labeled DON (A. Rahman et al., Phase I study and clinical pharmacology of 6-diazo-5-oxo-L-norleucine (DON), Invest. New Drugs. 3 (1985) 369-374) and a microbiological assay (D. A. Cooney et al., DON, CONV and DONV-III. Pharmacologic and toxicologic studies, Biochem. Pharmacol. 25 (1976) 1859-1870). HPLC analysis of DON suffers from interference from other materials in the sample; analysis may require boiling samples to confirm results (M. P. Sullivan et al., Pharmacokinetic and phase I study of intravenous DON (6-diazo-5-oxo-L-norleucine) in children, Cancer Chemother. Pharmacol. 21 (1988) 78-84) and often assays are not sensitive.

Using radiolabeled DON has the issues of working with radioactivity but more importantly, the assay does not differentiate intact DON from degraded DON or from metabolized or covalently bound DON that retains the radiolabel. Microbiological assays are time consuming, labor intensive, could suffer from nonspecific effects and may not differentiate between DON and its metabolites (D. A. Cooney et al., DON, CONV and DONV-III. Pharmacologic and toxicologic studies, Biochem. Pharmacol. 25 (1976) 1859-1870).

### SUMMARY

The presently disclosed subject matter provides methods for quantifying glutamine antagonists in a biological sample.

In one aspect, the presently disclosed subject matter provides a method for quantifying the amount of a glutamine antagonist in a biological sample, the method comprising: obtaining a biological sample comprising a glutamine antagonist; reacting the glutamine antagonist in the biological sample with an acidified alcohol to produce a derivatized glutamine antagonist; performing mass spectrometry (MS) to determine the amount of derivatized glutamine antagonist produced by the reaction; and comparing the amount of derivatized glutamine antagonist produced by the reaction to a standard curve to determine the amount of the glutamine antagonist in the biological sample. In particular aspects, the derivatized glutamine antagonist comprises:

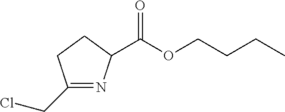

In another aspect, the presently disclosed subject matter provides a method for quantifying the amount of a glutamine antagonist in a biological sample, the method comprising: obtaining a biological sample comprising a glutamine antagonist; reacting the glutamine antagonist in the biological sample with an acidified alcohol to produce a derivatized glutamine antagonist comprising:

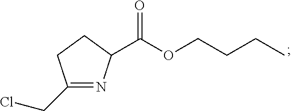

performing mass spectrometry (MS) to determine the amount of derivatized glutamine antagonist produced by the reaction; and comparing the amount of derivatized glutamine antagonist produced by the reaction to a standard curve to determine the amount of the glutamine antagonist in the biological sample.

In yet another aspect, the presently disclosed subject matter provides a method for quantifying the amount of a glutamine antagonist in a biological sample resulting from in vivo conversion of a prodrug of the glutamine antagonist to the glutamine antagonist, the method comprising: obtaining a biological sample comprising a glutamine antagonist resulting from in vivo conversion of a prodrug of the glutamine antagonist; reacting the glutamine antagonist in the biological sample with a chromophoric sulfonyl chloride under basic conditions to produce a derivatized glutamine antagonist; performing mass spectrometry (MS) to determine the amount of derivatized glutamine antagonist produced by the reaction; and comparing the amount of derivatized glutamine antagonist produced by the reaction to a standard curve to determine the amount of the glutamine antagonist in the biological sample resulting from in vivo conversion of the prodrug of the glutamine antagonist to the glutamine antagonist. In particular aspects, the derivatized glutamine antagonist comprises

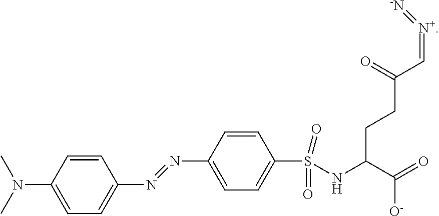

In still another aspect, the presently disclosed subject matter provides a method for quantifying the amount of a glutamine antagonist in a biological sample resulting from in vivo conversion of a prodrug of the glutamine antagonist to the glutamine antagonist, the method comprising: obtaining a biological sample comprising a glutamine antagonist resulting from in vivo conversion of a prodrug of the glutamine antagonist; reacting the glutamine antagonist in the biological sample with a chromophoric sulfonyl chloride under basic conditions to produce a derivatized glutamine antagonist comprising:

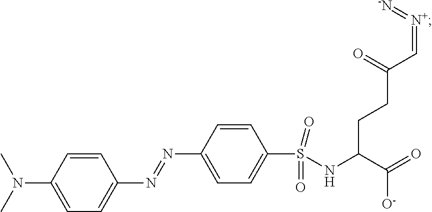

performing mass spectrometry (MS) to determine the amount of derivatized glutamine antagonist produced by the reaction; and comparing the amount of derivatized glutamine antagonist produced by the reaction to a standard curve to determine the amount of the glutamine antagonist in the biological sample resulting from in vivo conversion of the prodrug of the glutamine antagonist to the glutamine antagonist.

In other aspects, the presently disclosed subject matter provides a method for testing and/or monitoring the level of a glutamine antagonist in a subject, the method comprising: obtaining a biological sample comprising at least one glutamine antagonist from the subject; reacting the glutamine antagonist in the biological sample with acidified alcohol to produce a derivatized glutamine antagonist; performing mass spectrometry (MS) to determine the amount of derivatized glutamine antagonist produced by the reaction; and comparing the amount of derivatized glutamine antagonist produced by the reaction to a standard curve to determine the level of the glutamine antagonist in the subject. In particular aspects, the derivatized glutamine antagonist comprises

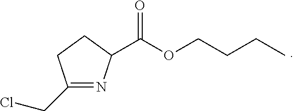

In still other aspects, the presently disclosed subject matter provides a method for testing and/or monitoring the level of a glutamine antagonist in a subject, the method comprising: obtaining a biological sample comprising at least one glutamine antagonist from the subject; reacting the glutamine antagonist in the biological sample with acidified alcohol to produce a derivatized glutamine antagonist comprising:

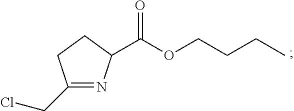

performing mass spectrometry (MS) to determine the amount of derivatized glutamine antagonist produced by the reaction; and comparing the amount of derivatized glutamine antagonist produced by the reaction to a standard curve to determine the level of the glutamine antagonist in the subject.

In particular aspects, the glutamine antagonist is 6-diazo-5-oxo-L-norleucine (DON).

In certain particular aspects, the acidified alcohol is butanol. In other particular aspects, the chromophoric sulfonyl chloride is dabsyl chloride. In yet more particular aspects, the biological sample comprises tissue and/or plasma.

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning. A Laboratory Manual, 3^{rd} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N.J., 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange 10^{th} ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), as of May 1, 2010, World Wide Web URL: http://www.ncbi.nlm.nih.gov/omim/ and in Online Mendelian Inheritance in Animals (OMIA), a database of genes, inherited disorders and traits in animal species (other than human and mouse), at http://omia.angis.org.au/contact.shtml.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIG. 1A, FIG. 1B, and FIG. 1C show: (FIG. 1A) derivatization of DON in 3N HCl plus n-butanol followed by LC-MS analysis. DON was heated at 60° C. for 30 min in n-butanol containing 3N HCl to derivatize the molecule into a stable quantifiable analyte of molecular mass [M+H]+ 218.0942; (FIG. 1B) the high resolution mass and isotopic abundance were used to generate the molecular formula C₁₀H₁₆ClNO₂. The observed isotopic abundance closely matched the predicted isotopic abundance of the proposed chemical structure incorporating a chlorine atom. This is apparent from the 3:1 ratio of [M] (218.0942) and [M+2] (220.0912); and (FIG. 1C) UPLC trace of derivatized DON. Derivatized DON was injected on an Agilent 1290 LC equipped with a C18 column and detected with an Agilent 6520 QTOF mass spectrometer;

FIG. 2 shows the derivatization of DON in 3N HCl without butanol followed by LC-MS analysis. The derivatized product exhibited a mass consistent with the proposed methylene chlorine substituted 1-pyrroline structure, but lacking the butyl ester;

FIG. 3A and FIG. 3B show tandem mass spectrometry (MS/MS) of derivatized DON: (FIG. 3A) collision induced dissociation of DON after derivatization with acidified butanol. DON was derivatized with n-butanol containing 3N HCl and analyzed by LC-MS/MS. The resulting product ions of 162.03 and 116.03 match the loss of the butyl ester and the radical formed after the loss of the entire carboxyl-ester moiety. These product ions are consistent with the expected DON derivative structure; and (FIG. 3B) collision induced dissociation of DON after derivatization with 3N HCl. DON was derivatized with 3N HCl without butanol and analyzed by LC-MS/MS. The resulting product ion of 116.0258 is consistent with the expected DON derivative structure in the absence of butanol;

FIG. 4A and FIG. 4B show: (FIG. 4A) standard curve of DON in plasma after derivatization with acidified butanol and LC-MS analysis. DON was spiked into untreated mouse plasma to generate standards at various concentrations. DON derivatization was carried out with n-butanol containing 3N HCl. After centrifugation to separate denatured proteins, the supernatant was incubated at 60° C. for 30 min. Derivatized DON was detected by LC-MS. Standards in the 30 nM to 100 μM range were used to generate a standard curve; and (FIG. 4B) standard curve of DON in brain after derivatization with acidified butanol and LC-MS analysis. DON was spiked into untreated mouse brain to generate standards at various concentrations. DON derivatization was carried out with n-butanol containing 3N HCl. After centrifugation to separate denatured proteins, supernatant was incubated at 60° C. for 30 min. Derivatized DON was detected by LC-MS. Standards in the 30 nM to 100 μM range were used to generate a standard curve;

FIG. 5A and FIG. 5B show: (FIG. 5A) DON concentrations in plasma over time. Mice were administered DON at 1.6 mg/kg (i.v.). Mice were euthanized and blood was collected transcardially 0.25, 0.5, 1, 2, 4 and 6 hours after dosing. Blood was centrifuged, plasma collected and stored at −80° C. N-butanol containing 3N HCl (250 μL) was added directly to samples (50 μL) and centrifuged at 16,000×g for 5 min to precipitate proteins. An aliquot (200 μL) of the supernatant was incubated at 60° C. for 30 min. After derivatization, the samples were analyzed by LC-MS (methods); and (FIG. 5B) DON plasma to brain ratio analysis. Mice were administered DON (0.6 mg/kg, i.p.) and blood and brain were collected 1 h after DON administration. Plasma was isolated from blood samples as described in (FIG. 5A). Brains were collected following perfusion with PBS and frozen immediately at −80° C. Before bioanalysis, brains were weighed and n-butanol containing 3N HCl was added to each sample (5 μL/mg tissue) and homogenized using a pestle. Resulting homogenates were centrifuged at 16,000×g for 5 min to precipitate proteins and analyzed the same way as the plasma samples. All determinations in both (A) and (B) were carried out in triplicate. Error bars correspond to ±S.E.M; and

FIG. 6 shows DON concentration in plasma from DON-treated mice using direct and derivatization protocols. Mouse plasma samples were obtained 15 min after DON administration (1.6 mg/kg, i.v.). For LC-MS bioanalysis of underivatized DON from plasma, DON was extracted from plasma with methanol, dried and resuspended in H₂O and separated in a Hypercarb column. For LC-MS bioanalysis of derivatized DON from plasma, DON was derivatized in butanol containing 3N HCl for 30 min at 60° C., dried, reconstituted in 30% acetonitrile and separated by RPC. Analytes eluting after each chromatographic separation were detected by QTOF MS. N=3 for each treatment. Error bars correspond to ±S.D.

### DETAILED DESCRIPTION

The presently disclosed subject matter now will be described more fully hereinafter with reference to the accompanying Examples and Figures, in which some, but not all embodiments of the presently disclosed subject matter are illustrated. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Examples and Figures. Therefore, it is to be understood that the presently disclosed subject matter is not to be limited to the specific embodiments disclosed and that modifications and other embodiments are intended to be included within the scope of the appended claims.

The presently disclosed subject matter provides a simple and robust method to quantify a glutamine antagonist, such as 6-diazo-5-oxo-L-norleucine (DON), in complex biological matrices by derivatizing the glutamine antagonist. Advantages of the presently disclosed methods include, but are not limited to, the use of a single solvent for extraction and derivatization which simplifies sample processing and shortens analysis time, unambiguous characterization of the derivatized product, and high sensitivity allowing a lower limit of quantitation than has been reported previously.

Previously, quantification of DON, an unstable polar compound, has been challenging. Derivatization of DON would not be expected to be successful because, in addition to the presence of a carboxylic acid moiety, there is the added complication of the diazo ketone moiety that lacks stability and is not expected to survive derivatization conditions.

The presently disclosed subject matter provides a bioanalytical method to quantify a glutamine antagonist by derivatizing the glutamine antagonist in acidified alcohol. Detection of the derivatized glutamine antagonist by mass spectrometry is fast, specific, and can be used to quantify the glutamine antagonist in biological samples, such as plasma and brain tissue, with a limit of detection to the low nanomolar level. The presently disclosed methods can be used in preclinical and clinical settings.

### I. Methods for Quantifying the Amount of a Glutamine Antagonist in a Biological Sample

In some embodiments, the presently disclosed subject matter provides a method for quantifying the amount of a glutamine antagonist in a biological sample, the method comprising: obtaining a biological sample comprising a glutamine antagonist; reacting the glutamine antagonist in the biological sample with an acidified alcohol to produce a derivatized glutamine antagonist; performing mass spectrometry (MS) to determine the amount of derivatized glutamine antagonist produced by the reaction; and comparing the amount of derivatized glutamine antagonist produced by the reaction to a standard curve to determine the amount of the glutamine antagonist in the biological sample. In particular embodiments, the derivatized glutamine antagonist comprises

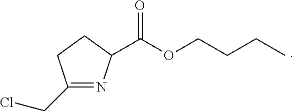

In some embodiments, the presently disclosed subject matter provides a method for quantifying the amount of a glutamine antagonist in a biological sample, the method comprising: obtaining a biological sample comprising a glutamine antagonist; reacting the glutamine antagonist in the biological sample with an acidified alcohol to produce a derivatized glutamine antagonist comprising:

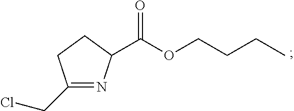

performing mass spectrometry (MS) to determine the amount of derivatized glutamine antagonist produced by the reaction; and comparing the amount of derivatized glutamine antagonist produced by the reaction to a standard curve to determine the amount of the glutamine antagonist in the biological sample.

As used herein, the term “glutamine antagonist” refers to glutamine analogs that can interfere with the ability of glutamine to function. By “analog” is meant a molecule that is not identical, but has analogous functional or structural features. Particularly, the glutamine antagonists used in the methods have the ability to be derivatized by an acidified alcohol. In particular embodiments, the glutamine antagonist is 6-diazo-5-oxo-L-norleucine (DON). In particular embodiments, the glutamine antagonist is acivicin (L-(alpha S,5S)-alpha-amino-3-chloro-4,5-dihydro-5-isoxazoleacetic acid). In particular embodiments, the glutamine antagonist is 5-diazo-4-oxo-L-norvaline (L-DONV). In particular embodiments, the glutamine antagonist is aza-serine. In some embodiments, the glutamine antagonist (e.g., glutamine analog) is selected from the group consisting of acivicin, DON, L-DONV, and aza-serine.

The presently disclosed subject matter also provides for quantification of prodrugs of glutamine antagonists or analogs.

In other embodiments, the presently disclosed subject matter provides a method for quantifying the amount of a glutamine antagonist in a biological sample resulting from in vivo conversion of a prodrug of the glutamine antagonist to the glutamine antagonist, the method comprising: obtaining a biological sample comprising a glutamine antagonist resulting from in vivo conversion of a prodrug of the glutamine antagonist; reacting the glutamine antagonist in the biological sample with a chromophoric sulfonyl chloride under basic conditions to produce a derivatized glutamine antagonist; performing mass spectrometry (MS) to determine the amount of derivatized glutamine antagonist produced by the reaction; and comparing the amount of derivatized glutamine antagonist produced by the reaction to a standard curve to determine the amount of the glutamine antagonist in the biological sample resulting from in vivo conversion of the prodrug of the glutamine antagonist to the glutamine antagonist. In particular embodiments, the derivatized glutamine antagonist comprises

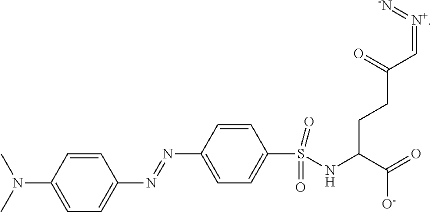

In other embodiments, the presently disclosed subject matter provides a method for quantifying the amount of a glutamine antagonist in a biological sample resulting from in vivo conversion of a prodrug of the glutamine antagonist to the glutamine antagonist, the method comprising: obtaining a biological sample comprising a glutamine antagonist resulting from in vivo conversion of a prodrug of the glutamine antagonist; reacting the glutamine antagonist in the biological sample with a chromophoric sulfonyl chloride under basic conditions to produce a derivatized glutamine antagonist comprising:

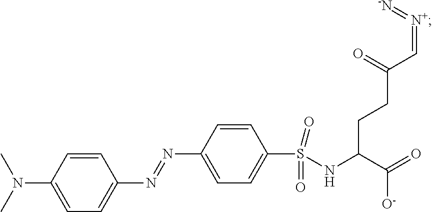

performing mass spectrometry (MS) to determine the amount of derivatized glutamine antagonist produced by the reaction; and comparing the amount of derivatized glutamine antagonist produced by the reaction to a standard curve to determine the amount of the glutamine antagonist in the biological sample resulting from in vivo conversion of the prodrug of the glutamine antagonist to the glutamine antagonist.

The presently disclosed subject matter contemplates derivatizing glutamine antagonists resulting from in vivo conversion of any prodrug of the glutamine antagonist to the glutamine antagonist, to provide for quantification of prodrugs of glutamine antagonists or analogs. Examples of suitable prodrugs of glutamine antagonists can be found in U.S. Provisional Application No. 62/199,566 filed on Jul. 31, 2015, which is incorporated herein by reference in its entirety. In particular embodiments, the prodrug of the glutamine antagonist is an ester prodrug of the glutamine antagonist. Exemplary ester prodrugs of the glutamine antagonist of use herein are also found in U.S. Provisional Application No. 62/199,566, including, for example, ester prodrugs of DON, acivicin, L-DONV, and aza-serine.

In the presently disclosed methods, the glutamine antagonist is obtained in a biological sample. The term “biological sample” encompasses a variety of sample types useful in the procedure of the presently disclosed subject matter. In one embodiment of the presently disclosed subject matter, the biological sample comprises tissue and/or plasma. In another embodiment, the tissue is brain tissue. Biological samples may include, but are not limited to, solid tissue samples, liquid tissue samples, biological fluids, aspirates, whole blood, hemocytes, serum, or cells and cell fragments. Specific examples of biological samples include, but are not limited to, solid tissue samples obtained by surgical removal, pathology specimens, archived samples, or biopsy specimens, tissue cultures or cells derived therefrom and the progeny thereof, and sections or smears prepared from any of these sources. Other examples of biological samples include any material derived from the body of a vertebrate animal, including, but not limited to, blood, cerebrospinal fluid, serum, plasma, urine, nipple aspirate, fine needle aspirate, tissue lavage such as ductal lavage, saliva, sputum, ascites fluid, liver, kidney, breast, bone, bone marrow, sciatic nerve, testes, brain, ovary, skin, lung, prostate, thyroid, pancreas, cervix, stomach, intestine, colorectal, brain, bladder, colon, nares, uterine, semen, lymph, vaginal pool, synovial fluid, spinal fluid, head and neck, nasopharynx tumors, amniotic fluid, breast milk, pulmonary sputum or surfactant, urine, fecal matter and other liquid samples of biologic origin.

After obtaining the biological sample comprising a glutamine antagonist, the glutamine antagonist is reacted with an acidified alcohol or chromophoric sulfonyl chloride to produce a derivatized glutamine antagonist. The term “acidified alcohol” refers to an alcohol that is in an acid, such as in hydrochloric acid (HCl). In some embodiments, the acidified alcohol is acidified butanol. In other embodiments, the acidified alcohol is in 3N HCl. In still other embodiments, reacting the glutamine antagonist in the biological sample with acidified alcohol comprises heating the glutamine antagonist with the acidified alcohol. In further embodiments, heating occurs for approximately 30 minutes. In still further embodiments, heating occurs at approximately 60° C. The term “chromophoric sulfonyl chloride” refers to a compound that contains a sulfonyl chloride moiety and a chromophore. In some embodiments, the chromophoric sulfonlyl chloride is dabyl chloride. In other embodiments, the chromophoric sulfonyl chloride is selected from the group consisting of dipsyl chloride, dabsyl chloride, lissamine rhodamine Beta sulfonyl chloride, pentafluorobenzene sulfonyl chloride, and combinations thereof. In some embodiments, the chromophoric sulfonyl chloride comprises a fluorophoric sulfonyl chloride, such as dansyl chloride. In some embodiments, the chromophoric sulfonyl chloride derivatizes the glutamine antagonist in the biological sample in the absence of hydrolyzing ester prodrugs of the glutamine antagonist in the biological sample. In further embodiments, heating occurs for approximately 15 minutes. In still further embodiments, heating occurs at approximately 60° C. In particular embodiments, the basic conditions comprise a buffer at a pH of 9. In particular embodiments, the basic conditions comprise a sodium bicarbonate buffer at a pH of 9. In further particular embodiments, the basic conditions comprise acetone. In certain embodiments, the glutamine antagonist is reacted with a chromophoric sulfonyl chloride in acetone and a sodium bicarbonate buffer at a pH of 9 by heating at 60° C. for approximately 15 minutes.

In some embodiments, the term “derivatized glutamine antagonist” as used herein refers to a glutamine antagonist that is derivatized to comprise the structure:

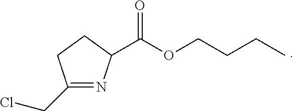

Those skilled in the art will appreciate that the structure of the derivatized glutamine antagonist shown above is the resulting structure for when DON is the glutamine antagonist derivatized with an acidified alcohol. Similarly, based on the chemistry of the derivization reaction and the guidance herein, those skilled in the art will be able to readily envision the structures of other glutamine antagonists derivatized with acidified alcohol, such as the structures of acivicin, L-DONV, and aza-serine derivitized with acidified alcohol, even though such structures are not shown herein.

In other embodiments, the term “derivatives glutamine antagonist” as used herein refers to a glutamine antagonist that is derivatized to comprise the structure:

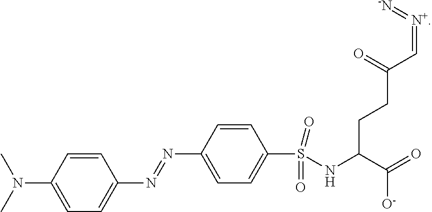

Those skilled in the art will appreciate that the structure of the derivatized glutamine antagonist shown above is the resulting structure for when DON is the glutamine antagonist derivatized with the chromophoric sulfonyl chloride dabsyl chloride. Similarly, based on the chemistry of the derivitization reaction and guidance herein, those skilled in the art will be able to readily envision the structures of other glutamine antagonists resulting from in vivo conversion of prodrugs of glutamine antagonists, such as acivicin, L-DONV, and aza-serine resulting from in vivo conversion of prodrugs of acivicin, L-DONV, and azaserine, derivitized with other chromphoric sulfonyl chlorides, such as dipsyl chloride, dabsyl chloride, lissamine rhodamine Beta sulfonyl chloride, pentafluorobenzene sulfonyl chloride.

After the derivatized glutamine antagonist is produced, mass spectrometry is used to determine the amount of derivatized glutamine antagonist produced by the reaction. In some embodiments, the mass spectrometry is liquid chromatography mass spectrometry (LC-MS) or liquid chromatography tandem mass spectrometry (LC MS/MS). In other embodiments, the method can be used to quantify the glutamine antagonist to levels as low as approximately 30 nM. In still other embodiments, the method can be used to quantify the glutamine antagonist, resulting from in vivo conversion of a prodrug of the glutamine antagonist to the glutamine antagonist, to levels as low as between approximately 50 nM and approximately 100 nM. In still other embodiments, the results from the mass spectrometry analysis are compared to a standard curve to determine the amount of the glutamine antagonist found in the biological sample.

### II. Methods for Testing and/or Monitoring the Levels of a Glutamine Antagonist in a Subject

In some embodiments, the presently disclosed subject matter provides methods for testing the levels of a glutamine antagonist in a subject. In some embodiments, the levels of a glutamine antagonist are tested in a subject more than once to monitor the levels over a period of time.

Accordingly, in some embodiments, the presently disclosed subject matter provides a method for testing and/or monitoring the level of a glutamine antagonist in a subject, the method comprising: obtaining a biological sample comprising a glutamine antagonist from the subject; reacting the glutamine antagonist in the biological sample with an acidified alcohol to produce a derivatized glutamine antagonist; performing mass spectrometry (MS) to determine the amount of derivatized glutamine antagonist produced by the reaction; and comparing the amount of derivatized glutamine antagonist produced by the reaction to a standard curve to determine the level of glutamine antagonist in the subject. In particular embodiments, the glutamine antagonist is 6-diazo-5-oxo-L-norleucine (DON). In particular embodiments, the derivatized glutamine antagonist comprises

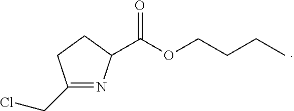

In some embodiments, the presently disclosed subject matter provides a method for testing and/or monitoring the level of a glutamine antagonist in a subject, the method comprising: obtaining a biological sample comprising a glutamine antagonist from the subject; reacting the glutamine antagonist in the biological sample with an acidified alcohol to produce a derivatized glutamine antagonist comprising:

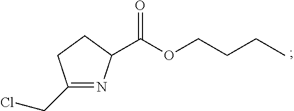

performing mass spectrometry (MS) to determine the amount of derivatized glutamine antagonist produced by the reaction; and comparing the amount of derivatized glutamine antagonist produced by the reaction to a standard curve to determine the level of glutamine antagonist in the subject. In particular embodiments, the glutamine antagonist is 6-diazo-5-oxo-L-norleucine (DON). In particular embodiments, the glutamine antagonist is acivicin. In particular embodiments, the glutamine antagonist is L-DONV. In particular embodiments, the glutamine antagonist is aza-serine. In some embodiments, the glutamine antagonist (e.g., glutamine analog) is selected from the group consisting of acivicin, DON, L-DONV, and aza-serine.

In other embodiments, the presently disclosed subject matter provides a method testing and/or monitoring the level of a glutamine antagonist in a subject resulting from in vivo conversion of a prodrug of the glutamine antagonist to the glutamine antagonist, the method comprising: obtaining from a subject a biological sample comprising a glutamine antagonist resulting from in vivo conversion in the subject of a prodrug of the glutamine antagonist to the glutamine antagonist; reacting the glutamine antagonist in the biological sample with a chromophoric sulfonyl chloride under basic conditions to produce a derivatized glutamine antagonist; performing mass spectrometry (MS) to determine the amount of derivatized glutamine antagonist produced by the reaction; and comparing the amount of derivatized glutamine antagonist produced by the reaction to a standard curve to determine the amount of the glutamine antagonist in the biological sample resulting from in vivo conversion of the prodrug of the glutamine antagonist to the glutamine antagonist. In particular embodiments, the derivatized glutamine antagonist comprises

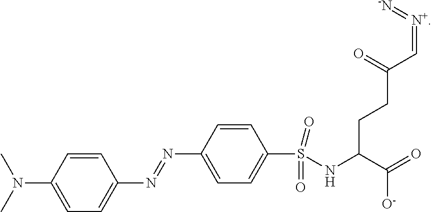

In other embodiments, the presently disclosed subject matter provides a method testing and/or monitoring the level of a glutamine antagonist in a subject resulting from in vivo conversion of a prodrug of the glutamine antagonist to the glutamine antagonist, the method comprising: obtaining from a subject a biological sample comprising a glutamine antagonist resulting from in vivo conversion in the subject of a prodrug of the glutamine antagonist to the glutamine antagonist; reacting the glutamine antagonist in the biological sample with a chromophoric sulfonyl chloride under basic conditions to produce a derivatized glutamine antagonist comprising:

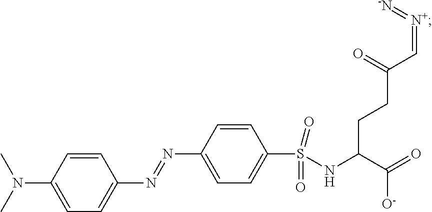

performing mass spectrometry (MS) to determine the amount of derivatized glutamine antagonist produced by the reaction; and comparing the amount of derivatized glutamine antagonist produced by the reaction to a standard curve to determine the amount of the glutamine antagonist in the biological sample resulting from in vivo conversion of the prodrug of the glutamine antagonist to the glutamine antagonist. In particular embodiments, the prodrug of the glutamine antagonist is an ester prodrug of the glutamine antagonist. In particular embodiments, the ester prodrug of the glutamine antagonist is an ester prodrug of acivicin. In particular embodiments, the ester prodrug of the glutamine antagonist is an ester prodrug of L-DONV. In particular embodiments, the ester prodrug of the glutamine antagonist is an ester prodrug of aza-serine.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal (non-human) subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. In some embodiments, the subject is human. In other embodiments, the subject is non-human. In still other embodiments, the biological sample comprises tissue and/or plasma. In further embodiments, the tissue is brain tissue.

In some embodiments, the acidified alcohol is acidified butanol. In other embodiments, the acidified alcohol is in 3N HCl. In still other embodiments, reacting the glutamine antagonist in the biological sample with an acidified alcohol comprises heating the glutamine antagonist with the acidified alcohol. In further embodiments, heating occurs for approximately 30 minutes. In still further embodiments, heating occurs at approximately 60° C.

In some embodiments, the chromophoric sulfonyl chloride is dabsyl chloride. In other embodiments, the chromophoric sulfonyl chloride is selected from the group consisting of dipsyl chloride, dabsyl chloride, lissamine rhodamine Beta sulfonyl chloride, pentafluorobenzene sulfonyl chloride, and combinations thereof. In further embodiments, reacting the glutamine antagonist in the biological sample with a chromophoric sulfonyl chloride comprises heating the glutamine antagonist with the chromophoric sulfonyl chloride under basic conditions. In further embodiments, heating occurs for approximately 15 minutes. In still further embodiments, heating occurs at approximately 60° C. In further embodiments, the basic conditions comprise a buffer at a pH of 9. In particular embodiments, the basic conditions comprise a sodium bicarbonate buffer at a pH of 9. In still even further embodiments, the basic conditions comprise acetone.

In some embodiments, the mass spectrometry to determine the amount of derivatized glutamine antagonist is liquid chromatography mass spectrometry (LC-MS) or liquid chromatography tandem mass spectrometry (LC MS/MS). In other embodiments, the method can be used to quantify the glutamine antagonist to levels as low as approximately 30 nM. In certain embodiments, the method can be used to quantify the glutamine antagonist, resulting from in vivo conversion of a prodrug of the glutamine antagonist to the glutamine antagonist, to levels as low as between approximately 50 nM and 100 nM.

In some embodiments, the presently disclosed methods further comprise administering the glutamine antagonist or a prodrug of the glutamine antagonist (e.g., an ester prodrug) to the subject before obtaining the biological sample from the subject. This may be a beneficial step when the glutamine antagonist is used as a tool compound in preclinical in vivo models or as a clinical candidate. The term “administering” as used herein refers to contacting a subject with a glutamine antagonist.

### III. General Definitions

Although specific terms are employed herein, they are used in a generic and descriptive sense only and not for purposes of limitation. Particular definitions are provided herein for clarity. Unless otherwise defined, all technical and scientific terms used herein have the same meaning as commonly understood by one of ordinary skill in the art to which this presently described subject matter belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

Any compositions or methods provided herein can be combined with one or more of any of the other compositions and methods provided herein.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Methods

DON Derivatization:

DON was derivatized in the presence of 3N HCl±n-butanol. DON (Sigma-Aldrich, St. Louis, Mo.) was first dissolved in water at a concentration of 10 mM. An aliquot (10 μL) of this stock solution was added to 3N HCl±n-butanol (250 μL) in a low retention micro-centrifuge tube. The solution was then heated at 60° C. for 30 minutes in a shaking water bath. After heating, the sample was dried at 45° C. under a nitrogen stream, resuspended in 50 μL of water/acetonitrile (70:30), vortexed and centrifuged at 16,000×g. Supernatants were transferred to LC vials and an aliquot (2 μL) was used for liquid chromatography mass spectrometry (LC-MS) or liquid chromatography tandem mass spectrometry (LC MS/MS) analysis.

DON Derivatization when Analyzing DON Resulting from Conversion of DON Prodrugs In Vivo:

When performing bioanalysis of DON in tissues resulting from conversion of DON prodrugs in vivo, acidic conditions used to derivatize DON could also hydrolyze prodrug moieties. This would make it difficult to differentiate DON converted from prodrug in vivo vs. DON converted from prodrug during sample preparation. Consequently, when analyzing for DON conversion from prodrugs, DON derivatization is carried out with dabsyl chloride which does not cause hydrolysis of esters. DON is extracted from approximately 50 mg samples with 5 μL methanol containing Glutamate-d5/mg tissue by pestle homogenization and vortexing in low retention tubes. Samples are centrifuged at 16,000×g for 5 min to precipitate proteins. Supernatants (200 μL) are moved to new tube and dried at 45° C. under vacuum for 1 h. To each tube, 50 μL of 0.2 M sodium bicarbonate buffer (pH 9.0) and 100 μL a 10 mM dabsyl chloride stock in acetone is added. After vortexing, samples are incubated at 60° C. for 15 minutes to derivatize (see scheme A below). Samples (2-10 μL) are injected and separated on an Agilent 1290 equipped with a SB-AQ column over a 4 minute gradient from 20-95% acetonitrile+0.1% formic acid and quantified on an Agilent 6520 QTOF mass spectrometer.

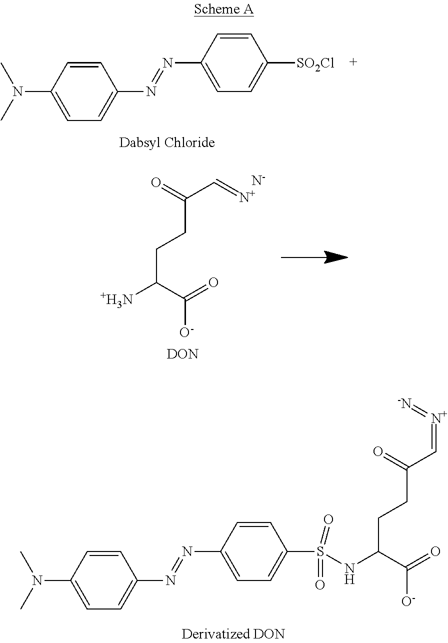

Analysis of Derivatized DON by LC-MS:

Derivatized DON samples (2 μL) prepared as described above were injected and separated on an Agilent 1290 LC equipped with an Agilent Eclipse Plus 2.1×100 mm, 1.8 micron Rapid Resolution C18 column over a 5.5 minute gradient from 30-70% acetonitrile+0.1% formic acid. Analytes were detected with an Agilent 6520 quadrupole time-of-flight (QTOF) mass spectrometer in positive mode with drying gas at 350° C., 11 L/min and 40 psi. The fragmenter was set at 70V and the VCAP at 4000V.

Analysis of Derivatized DON by LC-MS/MS:

Analysis of derivatized DON after 3N HCl±n-butanol by LC-MS/MS was carried out in the same manner as for LC-MS except the precursor mass (m/z=218.09 μL) was selected in the first quadrupole and the compound was made to collide with nitrogen gas with a collision energy of 15V in MS/MS mode to afford the daughter ions with m/z=162.032 and 116.026.

Analysis of Underivatized DON by LC-MS:

Methanol (250 μL) was added to plasma samples containing DON (50 μL); samples were centrifuged for 5 min at 16,000×g to precipitate proteins. An aliquot of the supernatant (200 μL) was dried and subsequently reconstituted in H₂O (50 μL). An aliquot (20 μL) was then injected and separated on an Agilent 1290 LC equipped with a Thermo Hypercarb 2.1×100 mm column with isocratic 2.5% acetonitrile+0.1% formic acid mobile phase. Analytes were detected with an Agilent 6520 QTOF in MS mode as when analyzing derivatized DON by LC-MS.

Bioanalysis of DON in Plasma:

When using plasma, DON was derivatized only using 3N HCl plus n-butanol and subsequently analyzed by LC-MS. To generate the standard curve to determine DON concentrations in plasma, DON (10 μL of 1 mM water solution) was added to untreated mouse plasma (90 μL) in a low retention micro-centrifuge tube. Standard solutions (100 μL) were then prepared by serial dilution to generate concentrations from 10 nM to 100 μM at half-log intervals. Prior to extraction, frozen plasma samples were thawed on ice. N-butanol (250 μL) containing 3N HCl was added directly to standards (50 μL), vortexed and centrifuged at 16,000×g for 5 minutes in low retention micro-centrifuge tubes to precipitate proteins. An aliquot (200 μL) of the supernatant was transferred to a new tube and incubated at 60° C. for 30 minutes in a shaking water bath to carry out the derivatization reaction. After derivatization, an aliquot of the reaction mixture (2 μL) was injected and analyzed by LC-MS as stated above. The area under the curve (AUC) representing the signal intensity of the extracted ion (m/z 218.0942) for each sample was used to generate the standard curve using Agilent Mass Hunter Quantitative analysis software. Plasma samples obtained from mice treated with DON were treated in exactly the same manner except exogenous DON was not added. DON concentrations in plasma samples were determined by interpolation using the standard curve.

Bioanalysis of DON in Brain:

When using brain, DON was derivatized only using 3N HCl plus n-butanol and subsequently analyzed by LC-MS. In order to generate the standard curve to determine DON concentrations in brain, frozen brain samples from untreated mice were thawed on ice. Tissue was weighed in low retention micro-centrifuge tubes to which 5 μL n-butanol containing 3N HCl were added per mg tissue. Tissue was then homogenized with a pestle and vortexed. Known amounts of DON from a 1 mM stock solution in water were mixed with n-butanol containing 3N HCl and spiked to brain tissue to prepare standards at concentrations from 10 nM to 100 μM at half-log intervals. Samples were centrifuged at 16,000×g for 5 minutes in low retention micro-centrifuge tubes to precipitate proteins. An aliquot (200 μL) of the supernatant was transferred to a new tube and incubated at 60° C. for 30 minutes in a shaking water bath to carry out the derivatization reaction. After derivatization, an aliquot of the reaction mixture (2 μL) was injected and analyzed by LC-MS as stated above. Brain samples obtained from mice treated with DON were treated in exactly the same manner except exogenous DON was not added. DON concentrations in brain samples were determined by interpolation using the standard curve.

Animal Studies:

All protocols were approved by the animal care and use committee at The Johns Hopkins University. C57BL/6 male mice (4-5 week old) after overnight fasting were administered DON at different doses either intravenously (i.v.) or intraperiotoneally (i.p.) as indicated. DON working solution was diluted in PBS each day from aliquots of a 100 mM stock solution in PBS stored at −80° C. At the indicated time points after DON administration, mice were euthanized in a CO₂ chamber and blood was collected transcardially. When collecting brains, mice were perfused with PBS before brain collection. Samples were frozen immediately at −80° C. and kept frozen until time for bioanalysis. Plasma and brain samples were processed and analyzed as stated in the bioanalysis of DON in plasma and brain sections.

### Example 2

### Results

LC-MS Analysis after DON Derivatization in 3N HCl n-Butanol Shows the Presence of a Chlorine-Containing Derivative:

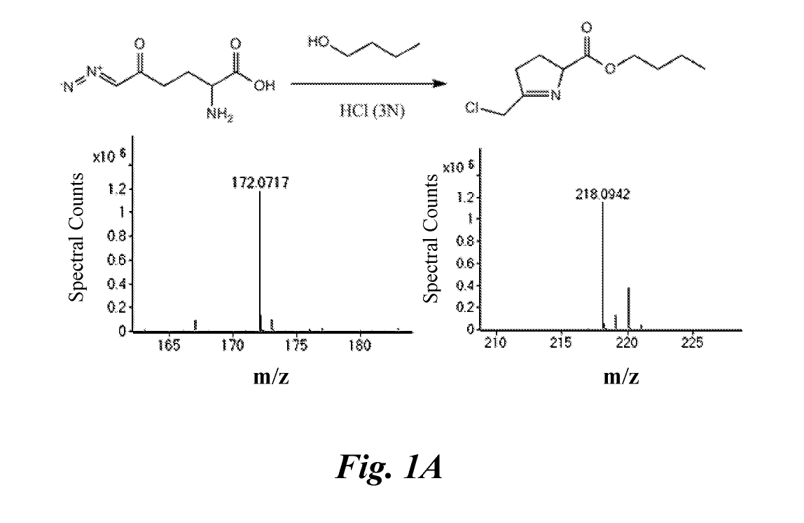

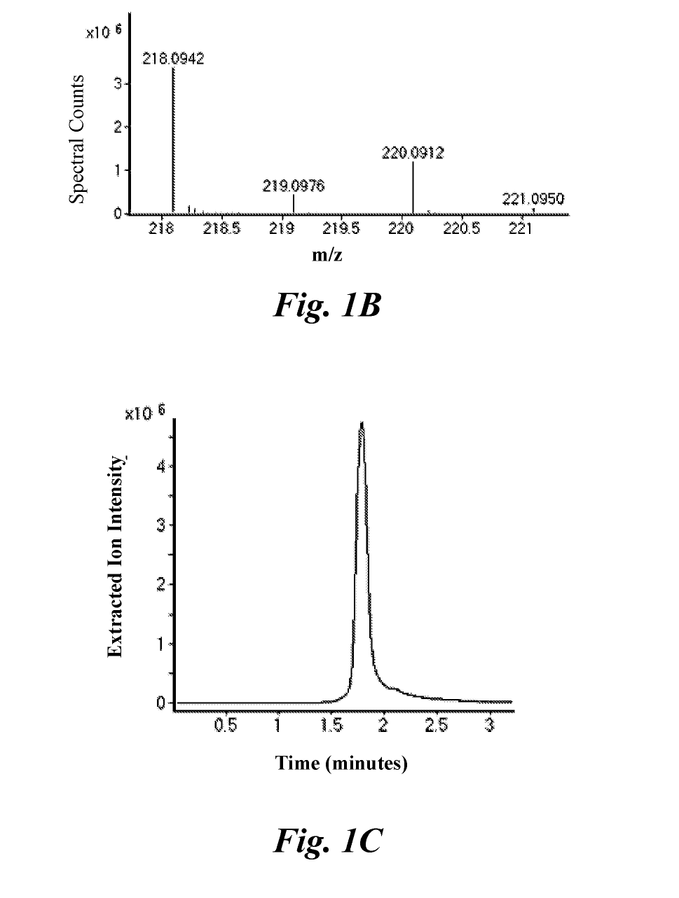

During DON derivatization using 3N HCl plus n-butanol, it was found that the diazo ketone moiety reacted and rearranged to form a stable and quantifiable derivative (FIG. 1A). The high resolution mass and the isotopic abundance were used to generate the molecular formula C₁₀H₁₆ClNO₂. The observed isotopic abundance closely matched the predicted isotopic abundance of the proposed chemical structure. The 3:1 isotopic abundance ratio between the molecular ion (218.0942) and the M+2 (220.0912) unambiguously indicated that a chlorine atom had been incorporated into the derivatized product (FIG. 1B). The corresponding chromatographic trace of the molecular ion of derivatized DON is shown in FIG. 1C. When DON was incubated with 3N HCl in water in the absence of butanol, a peak with molecular mass 162.0316 m/z was observed in the mass spectrum (FIG. 2). The molecular formula generated included a chlorine-containing derivative: C₆H₈ClNO₂. The structure that fits the molecular mass and molecular formula is the same as the derivatized structure in FIG. 1A without the butyl moiety (FIG. 2). This is as expected since butanol was not used in this derivatization reaction.

LC-MS/MS Analysis of Fragmentation Pattern of Ester-Containing Derivative Confirms the Presence of Cyclic Structure and Chlorine Atom:

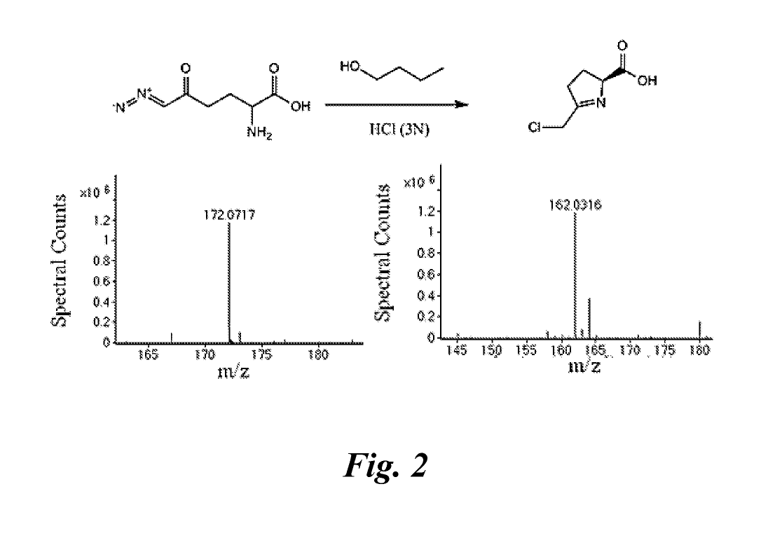

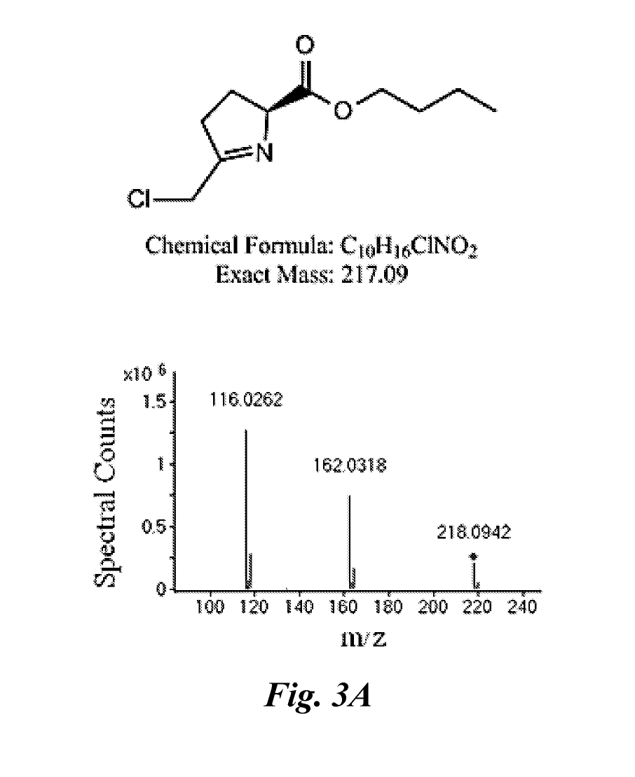

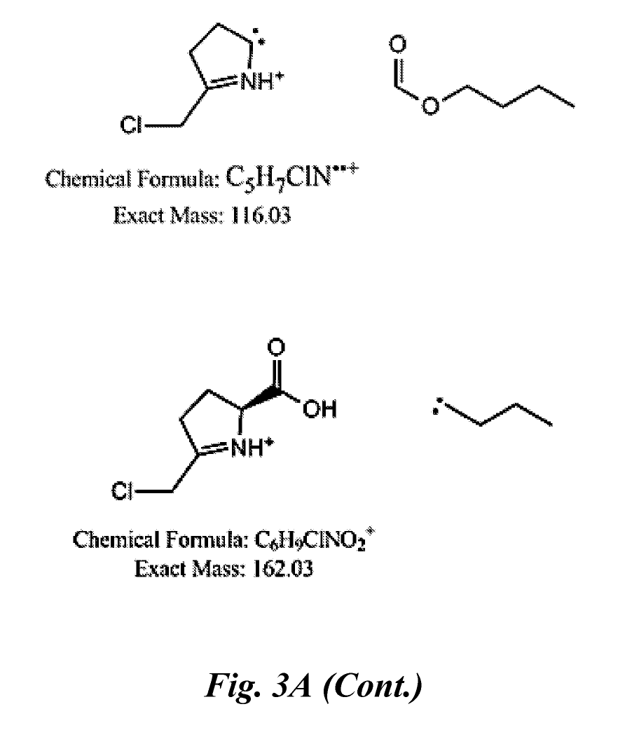

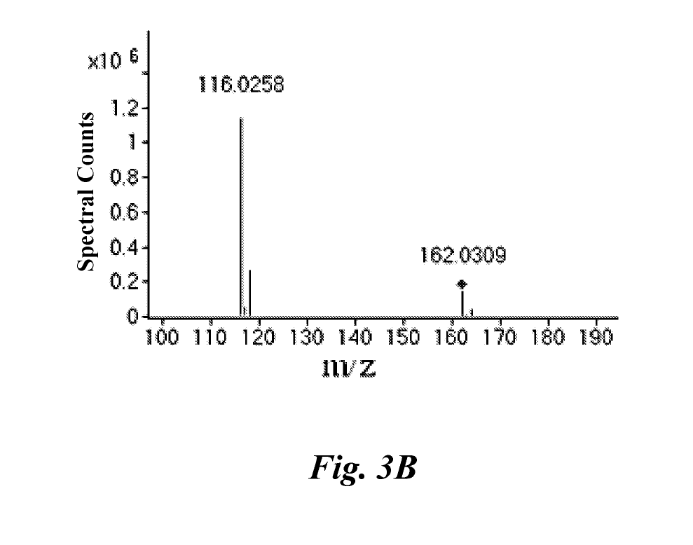

In a separate experiment, DON was derivatized with 3N HCl±n-butanol and subsequently analyzed by LC-MS/MS. The resulting product ions of 162.0318 and 116.0262 match the loss of the butyl ester and a radical formed after the loss of the entire carboxylate-ester moiety respectively (FIG. 3A). These product ions are consistent with the expected DON derivative structure. When using 3N HCl during derivatization, the resulting product ion of 116.0258 is consistent with the expected DON derivative structure in the absence of butanol (FIG. 3B).

DON Derivative was Quantified from Plasma and Brain Tissue Using LC-MS:

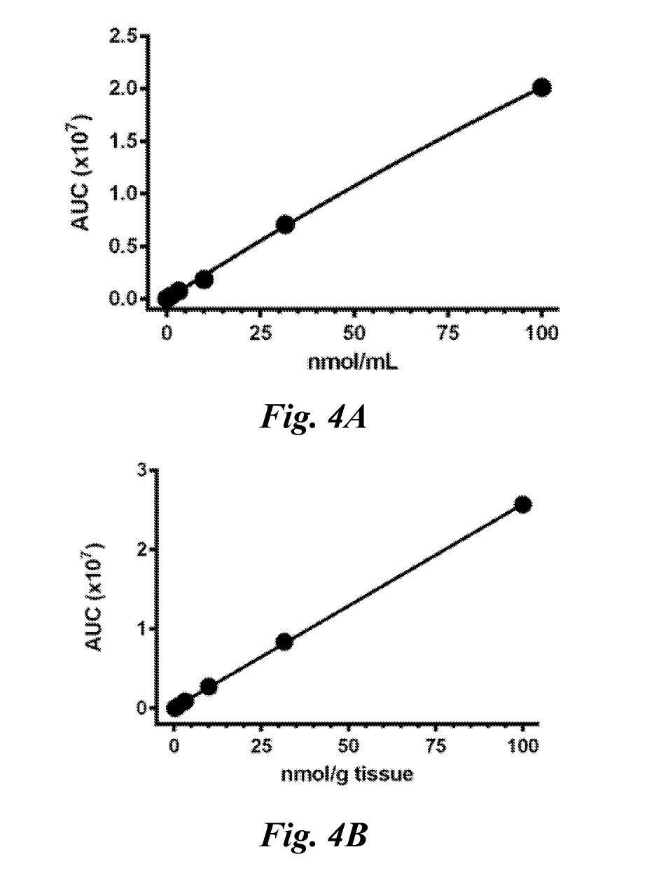

In order to verify that the DON derivatization protocol was adequate to use to determine DON concentrations in biological matrices, known concentrations of DON were added to mouse plasma and brain followed by derivatization using 3N HCl in n-butanol. Derivatized samples were then analyzed by LC-MS and a standard curve for each matrix was generated. In each case there was a linear correlation between signal response and the concentration of derivatized material. Standard curves were linear in the 30 nM-100 μM range for both plasma (FIG. 4A) and brain (FIG. 4B).

DON was Measured in Plasma and Brain Using the New Bioanalysis Procedure:

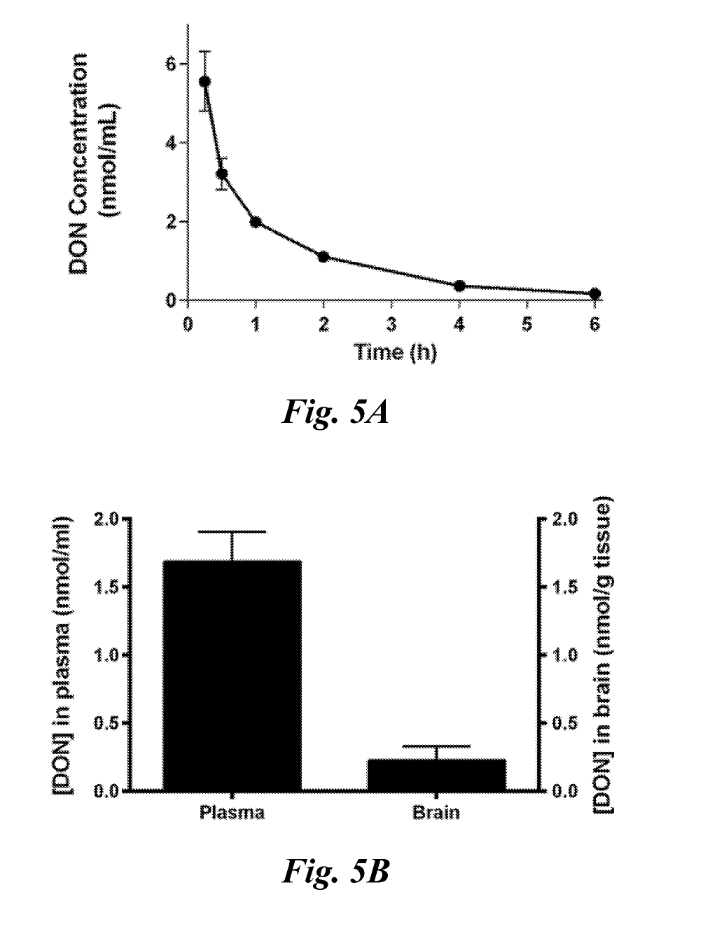

The new derivatizing procedure was used to determine DON concentrations in plasma and brain following i.v. and i.p. administration. In the first study, mice were given DON (1.6 mg/kg, i.v.) and blood was collected at 0.25, 0.5, 1, 2, 4 and 6 h. The exposure of DON estimated from the area under the curve (AUC) was 8 nmol h/mL with a plasma half-life of 1.2 h (FIG. 5A). In the second study, mice were given DON (0.6 mg/kg, i.p.) and both plasma and brain were harvested 1 h after DON administration. DON concentrations in plasma and brain were 1.7±0.5 μM and 0.22±0.18 nmol/g tissue respectively (FIG. 5B), suggesting a brain to plasma ratio of approximately 0.1.

Analysis of Derivatized DON or Intact DON Gave the Same Results:

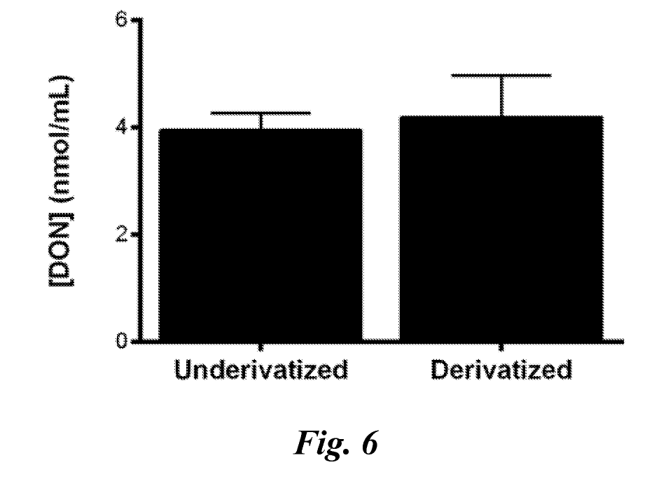

It is conceivable that when DON is used in vivo, it could form byproducts that could also form the derivatized structure. To determine if this was the case, DON concentration was measured both directly using a less sensitive method (LOD>1 μM) and through acidified butanol derivatization in plasma samples collected from mice 15 min after DON administration (1.6 mg/kg i.v.). The two methods gave the same DON concentration within experimental error: 3.9 μM±0.3 and 4.2 μM±0.8 when using the direct and derivatization methods respectively (FIG. 6).

### Example 3

### Discussion

Several quantification methods for DON have been previously described (M. P. Sullivan et al., Pharmacokinetic and phase I study of intravenous DON (6-diazo-5-oxo-L-norleucine) in children, Cancer Chemother. Pharmacol. 21 (1988) 78-84; C. Mueller et al., A phase IIa study of PEGylated glutaminase (PEG-PGA) plus 6-diazo-5-oxo-L-norleucine (DON) in patients with advanced refractory solid tumors J. Clin. Oncol. 26 (2008) 2533; B. B. Cao et al., The hypothalamus mediates the effect of cerebellar fastigial nuclear glutamatergic neurons on humoral immunity, Neuro. Endocrinol. Lett. 33 (2012) 393-400; L. M. Shelton et al., Glutamine targeting inhibits systemic metastasis in the VM-M3 murine tumor model, Int. J. Cancer. 127 (2010) 2478-2485) all with limits of detection in the low micromolar level.

HPLC/fluorescence, radiolabel and microbiological assays all have the potential for nonspecific signals. DON is a polar amino acid that elutes unretained in the void volume with many other polar compounds during reverse phase chromatography (RPC). Due to ion suppression and poor chromatography that result in broad irregular peak shapes, DON cannot be successfully separated and quantified from complex matrices such as brain and plasma with ordinary RPC. Direct measurement of DON quantification by LC/MS has been possible by using a porous graphitic carbon-based chromatographic column (Hypercarb) that minimizes the ion-suppression seen with the silica-based C18 column (unpublished observation); this measurement, however, also exhibits low sensitivity (LOD>1 μM) so it is not an alternative for routine pharmacokinetics samples where low nanomolar levels are of interest.

Polar amino acids are often derivatized to make them more amenable to separation by RPC (Molnar-Perl, (Ed.) Quantitation of Amino Acids and Amines by Chromatography: Methods and Protocols Elsevier (2005)). Esterification of the carboxylic acid on an amino acid improves RPC separation and increases the analyte mass which enhances ionization at the electrospray source of the mass spectrometer. For example, derivatization with an n-butyl ester has been used to quantify plasma methylmalonic acid (M. M. Kushnir et al., Analysis of dicarboxylic acids by tandem mass spectrometry. High-throughput quantitative measurement of methylmalonic acid in serum, plasma, and urine, Clin. Chem. 47 (2001) 1993-2002). In the case of DON, however, in addition to the presence of a carboxylic acid moiety, there is the added complication of the diazo ketone moiety that lacks stability and is not expected to survive derivatization conditions.

In an effort to develop a reliable way to measure DON in complex biological matrices, DON was incubated with butanol in 3N HCl for 30 min at 60° C., the same procedure used to derivatize carboxylic acids to make the corresponding n-butyl ester (M. M. Kushnir et al., Analysis of dicarboxylic acids by tandem mass spectrometry. High-throughput quantitative measurement of methylmalonic acid in serum, plasma, and urine, Clin. Chem. 47 (2001) 1993-2002). DON derivatization under these conditions produced a chlorine containing derivative as supported by the 3:1 isotopic abundance ratio between the molecular ion (218.0942) and the M+2 (220.0912) (FIG. 1B). Incorporation of a chloromethyl ketone to the diazo moiety of DON has been shown previously (B. Walker et al., Inhibition of Escherichia coli glucosamine synthetase by novel electrophilic analogues of glutamine-comparison with 6-diazo-5-oxo-norleucine, Bioorg. Med. Chem. Lett. 10 (2000) 2795-2798). The molecular mass (218.0942) (FIG. 1A) and isotopic abundances (FIG. 1B) observed in the mass spectrum are consistent with cyclization to form butyl 5-(chloromethyl)-3,4-dihydro-2H-pyrrole-2-carboxylate (FIG. 1A). When derivatization was carried out in 3N HCl in the absence of butanol, a chlorine-containing derivative that corresponds to the same cyclized product lacking the butyl group on the ester with molecular formula C₆H₈ClNO₂ (molecular mass 162.0316 m/z) was formed (FIG. 2).

In a separate effort to confirm the structure of derivatized DON, the product of derivatization was analyzed after collision induced dissociation (CID). The fragmentation pattern was consistent with the presence of an ester-containing derivative, and a 1-pyrroline ring with a methylene chlorine substitution (FIG. 3A). The resulting product ions match the loss of the butyl ester and a radical formed after the loss of the carboxylate-ester (FIG. 3A). Further confirmation was obtained when the 116.0258 product ion also was seen with CID of derivatized DON with 3N HCl without butanol (FIG. 3B).

A possible mechanism of the derivatization reaction in the absence of butanol is illustrated in Scheme B. At low pH (3N HCl), the α-carbon of the carbonyl close to the diazo moiety will abstract a proton from the solvent resulting in a diazonium ion. In the next step, the same α-carbon undergoes chlorine ion addition and concomitant N₂ loss. The chloromethyl ketone undergoes cyclization and dehydration to form the 1-pyrrolinedine derivative (5-member ring 1-pyrrolinedine with the methylene chlorine substitution) illustrated in Scheme B. When the derivatization reaction is carried out in n-butanol containing 3N HCl, the carboxylic acid moiety also will undergo standard acid-catalyzed esterification of the carboxylate moiety with the n-butyl group (P. Sykes, A guidebook to mechanism in organic chemistry, Third edition ed., Longman Group Limited (1975)).

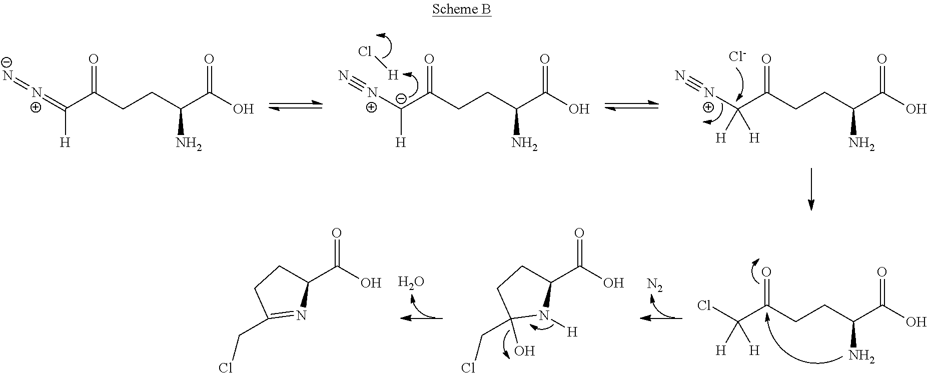

A new derivatizing procedure has been used to determine DON concentrations in both plasma and brain from mice after DON administration. First, standard curves were generated of signal intensity vs. known concentrations of DON that were added to both plasma and brain followed by derivatization, extraction and bioanalysis. The resulting standard curves for plasma (FIG. 4A) and brain (FIG. 4B) were then used to determine unknown concentrations of DON in plasma and brain from mice that had been treated with DON. The results show that DON can be readily monitored using the new derivatizing procedure. i.v. pharmacokinetic parameters of t_{1/2}=1.2 h and AUC=8 nmol h/mL were reported (FIG. 5A) and for the first time show DON brain penetration with a brain/plasma ratio of 10% at 1 h (FIG. 5B). The latter finding was surprising given DON's polar structure. Since the animals were perfused, DON found in brain at this level is unlikely to be due to blood contamination. DON could be actively transported by an amino acid carrier into the brain; previous reports have demonstrated active DON uptake systems that can be inhibited by glutamine in leukemia cells (K. R. Huber et al., Uptake of glutamine antimetabolites 6-diazo-5-oxo-L-norleucine (DON) and acivicin in sensitive and resistant tumor cell lines, Int. J. Cancer. 41 (1988) 752-755 and xenopus oocytes P. M. Taylor, et al., Transport and membrane binding of the glutamine analogue 6-diazo-5-oxo-L-norleucine (DON) in Xenopus laevis oocytes, J. Membr. Biol. 128 (1992) 181-191).

One potential drawback of the bioanalysis procedure is that DON could cyclize in vivo to form a byproduct, which in turn could convert into the analyte during derivatization. This could give artificially high DON concentrations. To rule out this possibility, a control experiment was performed where DON concentrations obtained when using the derivatizing protocol (3N HCl+n-butanol) and when measuring DON directly were compared. Even though direct measurement of DON is far less sensitive than when using the derivatizing procedure (LOD for direct method >1 μM vs. LOD for derivatizing procedure=30 nM), when measuring μM levels of DON, a side by side comparison of the two methods would unveil whether the derivatization method is measuring DON byproducts. It was found that the derivatization procedure gave the same DON concentrations within error as the direct measurements of underivatized DON (FIG. 6). Further, a cyclized byproduct, made by heating DON at 37° C. for 2 h then added to plasma did not convert to the derivatized product when using the derivatization protocol. The result showed the cyclized byproduct of DON was impervious to the derivatization procedure. In a separate study, the cyclized byproduct of DON was not observed by LC-MS analysis of plasma from DON-treated mice 2 h after i.v. treatment.

In summary, a simple and robust method has been developed to quantify DON in complex biological matrices using UPLC/MS after DON derivatization with acidified butanol. DON in the sample is made to react with n-butanol containing 3 N HCl to form butyl 5-(chloromethyl)-3,4-dihydro-2H-pyrrole-2-carboxylate. A single solvent for extraction and derivatization solution simplifies sample processing and shortens analysis time. The derivatization LC/MS method is rapid, reproducible and rigorous and has a lower limit of quantitation of 30 nM that is over 30-fold more sensitive than methods reported in the literature. Mass spectrometry is able to reduce nonspecific signal, since only one analyte with a specific molecular formula is quantified. This method was applied to monitor DON levels in plasma and brain and could readily be applied to other tissues as well.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
J. G. Cory, and A. H. Cory, Critical roles of glutamine as nitrogen donors in purine and pyrimidine nucleotide synthesis: asparaginase treatment in childhood acute lymphoblastic leukemia, In Vivo. 20 (2006) 587-589.
X. Tong, F. Zhao, and C. B. Thompson, The molecular determinants of de novo nucleotide biosynthesis in cancer cells, Curr Opin Genet Dev. 19 (2009) 32-37.
R. J. DeBerardinis, and T. Cheng, Q's next: the diverse functions of glutamine in metabolism, cell biology and cancer, Oncogene. 29 (2010) 313-324.
D. L. Kisner, R. Catane, and F. M. Muggia, The rediscovery of DON (6-diazo-5-oxo-L-norleucine), Recent Results Cancer Res. 74 (1980) 258-263.
A. Le, A. N. Lane, M. Hamaker, S. Bose, A. Gouw, J. Barbi, T. Tsukamoto, C. J. Rojas, B. S. Slusher, H. Zhang, L. J. Zimmerman, D. C. Liebler, R. J. Slebos, P. K. Lorkiewicz, R. M. Higashi, T. W. Fan, and C. V. Dang, Glucose-independent glutamine metabolism via TCA cycling for proliferation and survival in B cells, Cell Metab. 15 (2012) 110-121.
C. M. Thanki, D. Sugden, A. J. Thomas, and H. F. Bradford, In vivo release from cerebral cortex of [14C]glutamate synthesized from [U-14C]glutamine, J Neurochem. 41 (1983) 611-617.
P. Marmiroli, and G. Cavaletti, The glutamatergic neurotransmission in the central nervous system, Curr Med Chem. 19 (2012) 1269-1276.
T. W. Lai, S. Zhang, and Y. T. Wang, Excitotoxicity and stroke: identifying novel targets for neuroprotection, Prog Neurobiol. 115 (2013) 157-188.
S. Vucic, and M. C. Kiernan, Utility of transcranial magnetic stimulation in delineating amyotrophic lateral sclerosis pathophysiology, Handb Clin Neurol. 116 (2013) 561-575.
M. D. Sepers, and L. A. Raymond, Mechanisms of synaptic dysfunction and excitotoxicity in Huntington's disease, Drug Discov Today (201 μL).
M. R. Hynd, H. L. Scott, and P. R. Dodd, Glutamate-mediated excitotoxicity and neurodegeneration in Alzheimer's disease, Neurochem Int. 45 (200 μL) 583-595.
M. C. Potter, M. Figuera-Losada, C. Rojas, and B. S. Slusher, Targeting the glutamatergic system for the treatment of HIV-associated neurocognitive disorders, J Neuroimmune Pharmacol. 8 (2013) 594-607.
C. J. Chen, Y. C. Ou, C. Y. Chang, H. C. Pan, S. L. Liao, S. Y. Chen, S. L. Raung, and C. Y. Lai, Glutamate released by Japanese encephalitis virus-infected microglia involves TNF-alpha signaling and contributes to neuronal death, Glia. 60 (2012) 487-501.
A. R. Jayakumar, K. V. Rao, R. Murthy Ch, and M. D. Norenberg, Glutamine in the mechanism of ammonia-induced astrocyte swelling, Neurochem Int. 48 (2006) 623-628.
I. Maezawa, and L. W. Jin, Rett syndrome microglia damage dendrites and synapses by the elevated release of glutamate, J Neurosci. 30 (2010) 5346-5356.
A. G. Thomas, C. M. O'Driscoll, J. Bressler, W. Kaufmann, C. J. Rojas, and B. S. Slusher, Small molecule glutaminase inhibitors block glutamate release from stimulated microglia, Biochem Biophys Res Commun. 443 (2014) 32-36.
C. Tian, N. Erdmann, J. Zhao, Z. Cao, H. Peng, and J. Zheng, HIV-infected macrophages mediate neuronal apoptosis through mitochondrial glutaminase, J Neurochem. 105 (2008) 994-1005.
S. C. Hartman, and T. F. McGrath, Glutaminase A of escherichia coli. Reactions with the substrate analogue, 6-diazo-5-oxonorleucine, J Biol Chem. 248 (1973) 8506-8510.
R. A. Shapiro, V. M. Clark, and N. P. Curthoys, Inactivation of rat renal phosphate-dependent glutaminase with 6-diazo-5-oxo-L-norleucine. Evidence for interaction at the glutamine binding site, J Biol Chem. 254 (1979) 2835-2838.
K. Thangavelu, Q. Y. Chong, B. C. Low, and J. Sivaraman, Structural basis for the active site inhibition mechanism of human kidney-type glutaminase (KGA), Sci Rep. 4 (2011) 3827.
R. H. Earhart, J. M. Koeller, and H. L. Davis, Phase I trial of 6-diazo-5-oxo-L-norleucine (DON) administered by 5-day courses, Cancer Treat Rep. 66 (1982) 1215-1217.
J. S. Kovach, R. T. Eagan, G. Powis, J. Rubin, E. T. Creagan, and C. G. Moertel, Phase I and pharmacokinetic studies of DON, Cancer Treat Rep. 65 (1981) 1031-1036.
G. Lynch, N. Kemeny, and E. Casper, Phase II evaluation of DON (6-diazo-5-oxo-L-norleucine) in patients with advanced colorectal carcinoma, Am J Clin Oncol. 5 (1982) 541-543.
J. Rubin, S. Sorensen, A. J. Schutt, G. A. van Hazel, M. J. O'Connell, and C. G. Moertel, A phase II study of 6-diazo-5-oxo-L-norleucine (DON, NSC-7365) in advanced large bowel carcinoma, Am J Clin Oncol. 6 (1983) 325-326.
R. B. Sklaroff, E. S. Casper, G. B. Magill, and C. W. Young, Phase I study of 6-diazo-5-oxo-L-norleucine (DON), Cancer Treat Rep. 64 (1980) 1247-1251.
M. P. Sullivan, J. A. Nelson, S. Feldman, and B. Van Nguyen, Pharmacokinetic and phase I study of intravenous DON (6-diazo-5-oxo-L-norleucine) in children, Cancer Chemother Pharmacol. 21 (1988) 78-84.
C. Mueller, S. Al-Batran, E. Jaeger, B. Schmidt, M. Bausch, C. Unger, and N. Sethuraman, A phase IIa study of PEGylated glutaminase (PEG-PGA) plus 6-diazo-5-oxo-L-norleucine (DON) in patients with advanced refractory solid tumors Journal of Clinical Oncology 26 (2008) 2533.
B. B. Cao, X. H. Han, Y. Huang, Y. H. Qiu, and Y. P. Peng, The hypothalamus mediates the effect of cerebellar fastigial nuclear glutamatergic neurons on humoral immunity, Neuro Endocrinol Lett. 33 (2012) 393-400.
L. M. Shelton, L. C. Huysentruyt, and T. N. Seyfried, Glutamine targeting inhibits systemic metastasis in the VM-M3 murine tumor model, Int J Cancer. 127 (2010) 2478-2485.
G. Powis, and M. M. Ames, Determination of 6-diazo-5-oxo-L-norleucine in plasma and urine by reversed-phase high-performance liquid chromatography of the dansyl derivative, J Chromatogr. 181 (1980) 95-99.
J. A. Nelson, and B. Herbert, Rapid Analysis of 6Diazo5-oxo-L-norleucine (DON) in Human Plasma and Urine, Journal of Liquid Chromatography & Related Technologies 4(1981) 1641-1649.
A. Rahman, F. P. Smith, P. T. Luc, and P. V. Woolley, Phase I study and clinical pharmacology of 6-diazo-5-oxo-L-norleucine (DON), Invest New Drugs. 3 (1985) 369-374.
D. A. Cooney, H. N. Jayaram, H. A. Milman, E. R. Homan, R. Pittillo, Geran, J. Ryan, and R. J. Rosenbluth, DON, CONV and DONV-III. Pharmacologic and toxicologic studies, Biochem Pharmacol. 25 (1976) 1859-1870.
Molnár-Perl, (Ed.) Quantitation of Amino Acids and Amines by Chromatography: Methods and Protocols Elsevier (2005).
M. M. Kushnir, G. Komaromy-Hiller, B. Shushan, F. M. Urry, and W. L.

Roberts, Analysis of dicarboxylic acids by tandem mass spectrometry. High-throughput quantitative measurement of methylmalonic acid in serum, plasma, and urine, Clin Chem. 47 (2001) 1993-2002.
B. Walker, M. F. Brown, J. F. Lynas, S. L. Martin, A. McDowell, B. Badet, and A. J. Hill, Inhibition of Escherichia coli glucosamine synthetase by novel electrophilic analogues of glutamine—comparison with 6-diazo-5-oxo-norleucine, Bioorg Med Chem Lett. 10 (2000) 2795-2798.
P. Sykes, A guidebook to mechanism in organic chemistry, Third edition ed., Longman Group Limited (1975).
K. R. Huber, H. Rosenfeld, and J. Roberts, Uptake of glutamine antimetabolites 6-diazo-5-oxo-L-norleucine (DON) and acivicin in sensitive and resistant tumor cell lines, Int J Cancer. 41 (1988) 752-755.
P. M. Taylor, B. Mackenzie, H. S. Hundal, E. Robertson, and M. J. Rennie, Transport and membrane binding of the glutamine analogue 6-diazo-5-oxo-L-norleucine (DON) in Xenopus laevis oocytes, J Membr Biol. 128 (1992) 181-191.

Although the foregoing subject matter has been described in some detail by way of illustration and example for purposes of clarity of understanding, it will be understood by those skilled in the art that certain changes and modifications can be practiced within the scope of the appended claims.

1. A method for quantifying the amount of a glutamine antagonist in a biological sample, the method comprising:
obtaining a biological sample comprising a glutamine antagonist;
reacting the glutamine antagonist in the biological sample with an acidified alcohol to produce a derivatized glutamine antagonist;
performing mass spectrometry (MS) to determine the amount of derivatized glutamine antagonist produced by the reaction; and
comparing the amount of derivatized glutamine antagonist produced by the reaction to a standard curve to determine the amount of the glutamine antagonist in the biological sample.

2. The method of claim 1, wherein the acidified alcohol is selected from the group consisting of acidified butanol and 3N hydrochloric acid (HCl).

3. (canceled)

4. The method of claim 1, wherein the glutamine antagonist is selected from the group consisting of acivicin (L-(alpha S,5S)-alpha-amino-3-chloro-4,5-dihydro-5-isoxazoleacetic acid), 6-diazo-5-oxo-norleucine (DON), and 5-diazo-4-oxo-L-norvaline (L-DONV), and aza-serine.

5. The method of claim 1, wherein the derivatized glutamine antagonist comprises:

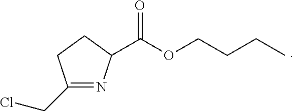

6. The method of claim 1, wherein the biological sample comprises tissue and/or plasma.

7. The method of claim 6, wherein the tissue is brain tissue.

8. The method of claim 1, wherein the method can be used to quantify the glutamine antagonist to levels as low as approximately 30 nM.

9. The method of claim 1, wherein the mass spectrometry is liquid chromatography mass spectrometry (LC-MS) or liquid chromatography tandem mass spectrometry (LC MS/MS).

10. The method of claim 1, wherein reacting the glutamine antagonist in the biological sample with the acidified alcohol comprises heating the glutamine antagonist with the acidified alcohol.

11. The method of claim 10, wherein the heating occurs for approximately 30 minutes.

12. The method of claim 10, wherein the heating occurs at approximately 60° C.

13-26. (canceled)

27. A method for quantifying the amount of a glutamine antagonist in a biological sample resulting from in vivo conversion of a prodrug of the glutamine antagonist to the glutamine antagonist, the method comprising:
obtaining a biological sample comprising a glutamine antagonist resulting from in vivo conversion of a prodrug of the glutamine antagonist;
reacting the glutamine antagonist in the biological sample with a chromophoric sulfonyl chloride under basic conditions to produce a derivatized glutamine antagonist;
performing mass spectrometry (MS) to determine the amount of derivatized glutamine antagonist produced by the reaction; and
comparing the amount of derivatized glutamine antagonist produced by the reaction to a standard curve to determine the amount of the glutamine antagonist in the biological sample resulting from in vivo conversion of the prodrug of the glutamine antagonist to the glutamine antagonist.

28. The method of claim 27, wherein the chromophoric sulfonyl chloride is selected from the group consisting of dabsyl chloride, dipsyl chloride, diabsyl chloride, lissamine rhodamine Beta sulfonyl chloride, and pentafluorobenzene sulfonyl chloride.

29. The method of claim 27, wherein the chromophoric sulfonyl chloride is dabsyl chloride.

30. The method of claim 27, wherein the basic conditions comprise a buffer at a pH of 9.

31. The method of claim 27, wherein the basic conditions comprise a sodium bicarbonate buffer at a pH of 9.

32. The method of claim 27, wherein the basic conditions comprise acetone.

33. The method of claim 27, wherein the glutamine antagonist is selected from the group consisting of acivicin (L-(alpha S,5S)-alpha-amino-3-chloro-4,5-dihydro-5-isoxazoleacetic acid), 6-diazo-5-oxo-norleucine (DON), and 5-diazo-4-oxo-L-norvaline (L-DONV), and aza-serine.

34. The method of claim 27, wherein the prodrug of the glutamine antagonist is an ester prodrug of the glutamine antagonist.

35. The method of claim 27, wherein the derivatized glutamine antagonist comprises:

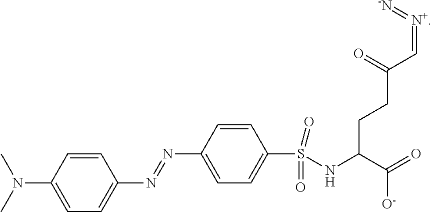

36. The method of claim 27, wherein the biological sample comprises tissue and/or plasma.

37. The method of claim 36, wherein the tissue is brain tissue.

38. The method of claim 27, wherein the method can be used to quantify the glutamine antagonist to levels as low as between approximately 50 nM and approximately 100 nM.

39. The method of claim 27, wherein the mass spectrometry is liquid chromatography mass spectrometry (LC-MS) or liquid chromatography tandem mass spectrometry (LC MS/MS).

40. The method of claim 27, wherein reacting the glutamine antagonist in the biological sample with the chromophoric sulfonyl chloride comprises heating the glutamine antagonist with the chromophoric sulfonyl chloride.

41. The method of claim 40, wherein the heating occurs for approximately 15 minutes.

42. The method of claim 40, wherein the heating occurs at approximately 60° C.

43. The method of claim 25, wherein the biological sample is obtained from a subject.

44. The method of claim 43, wherein quantifying the amount of the glutamine antagonist in a biological sample comprises testing and/or monitoring the level of a glutamine antagonist in the subject.

45. The method of claim 44, further comprising administering the prodrug of the glutamine antagonist to the subject prior to obtaining the biological sample.

46. The method of claim 27, wherein the chromophoric sulfonyl chloride derivatizes the glutamine antagonist in the biological sample in the absence of hydrolyzing ester prodrugs of the glutamine antagonist in the biological sample.

47. The method of claim 1, wherein the biological sample is obtained from a subject.

48. The method of claim 47, wherein the subject is human.

49. The method of claim 48, wherein quantifying the amount of the glutamine antagonist in the biological sample comprises testing and/or monitoring the level of a glutamine antagonist in the subject.

50. The method of claim 49, further comprising administering the glutamine antagonist to the subject prior to obtaining the biological sample.
